SEQUENCE LISTING (28 sequences)

SEQ ID NO: 1 - protein, 20 aa, Enterobacter aerogenes
  SIGNAL (1)..(20)
MKKRVLALCLASLFSVNAFA

SEQ ID NO: 2 - DNA, 60 bp, Enterobacter aerogenes
  sig_peptide (1)..(60)
atgaaaaagcgcgttctcgccctctgcctcgccagcctgttttccgttaacgctttcgcg

SEQ ID NO: 3 - protein, 594 aa, Empedobacter brevis
QDAKADSAYVRDNYEKIEQVIPMRDGTKLFTAIYQPKDKTKQYPVLLNRTPYTVAPYGVNEYKKSLGNFPTEMREGFIFVYQDVRGKWMSEGEFEDVRPINPSKSKKAIDESTDTFDTLEWLAKNLKNYTKKAGIYGISYPGFYSTMSLVNSHPTLKAVSPQAPVTNWFLGDDFHHNGVLFLNDSFSFMTFFGVKRPQPITPDKGPKRFEYPIKDNYRFYASGSVKELKDKYLQDNIKFYNDLFAHPDYDQFWQDRNVLPHLTNVQPAVMTVGGFFDAEDVYGAFETYKAIEKQNPKATNIMVAGPWFHGGWVRSNGSTFGDMQFASNTSEHYQQEIELPFFNYYLKDKGNFKPTEATIFITGSNEWKQFDAWPPKNVTTQKIYLQQNGKIAFNKTNTTTTFDEYVADPNSPVPYSGGVLETRSREYMVDDQRFASTRPDVMVYQSDILTEDITLAGPVINHLVVSTTGTDADYVVKLIDVYPENTPKFNNKLMAGYQNLIRAEIMRGKYRNSFSNPEAMVPNKETNVTYTMPDVGHTFKKGHRIMIQVQNSWFPLADRNPQQFMNVYEATSKDYLKQTQRIYHTSYIEIPVLK

SEQ ID NO: 4 - protein, 386 aa, Pseudomonas taetrolens
APPLSMTDGVAQVNTQDSNAWVEINKAAFEHNIRTLQTALAGKSQICAVLKADAYGHGIGLLMPSVIAMGVPCVGVASNEEARVVRESGFKGQLIRVRTAALSELEAALPYNMEELVGNLDFAVKASLIAEDHGRPLVVHLGLNSSGMSRNGVDMTTAQGRRDAVAITKVPNLEVRAIMTHFAVEDAADVRAGLKAFNQQAQWLMNVAQLDRSKITLHAANSFATLEVPESHLDMVRPGGALFGDTVPSHTEYKRVMQFKSHVASVNSYPKGNTVGYGRTYTLGRDSRLANITVGYSDGYRRAFTNKGIVLINGHRVPVVGKVSMNTLMVDVTDAPDVKSGDEVVLFGHQGKAEITQAEIEDINGALLADLYTVWGNSNPKILKDQ

SEQ ID NO: 5 - DNA, 30 bp, Artificial Sequence
  Synthetic DNA
cttacagatgactataatgtgactaaaaac

SEQ ID NO: 6 - DNA, 30 bp, Artificial Sequence
  Synthetic DNA
gtttttagtcacattatagtcatctgtaag

SEQ ID NO: 7 - protein, 9 aa, Empedobacter brevis
LFTAIYQPK

SEQ ID NO: 8 - protein, 9 aa, Empedobacter brevis
TNVTYTMPD

SEQ ID NO: 9 - DNA, 20 bp, Artificial Sequence
  Synthetic DNA
ttyacngcnathtaycarcc

SEQ ID NO: 10 - DNA, 23 bp, Artificial Sequence
  Synthetic DNA
tcnggcatngtrtangtnacrtt

SEQ ID NO: 11 - DNA, 2024 bp, Empedobacter brevis
  CDS (61)..(1908)
atttcttaataaaaactgaaatcttaatacatttatactatcgtaaaatttattgaacacgtgaaaaaattaacattaaaagtaactctacttacacttttgttgggaagtacagttggatttgcgcaagatgcaaaagcagattctgcttatgtgcgcgacaattacgaaaaaatagaacaagtaattccgatgcgcgatggtacaaagttatttacagctatttatcagccaaaagataaaacaaaacaatatcccgttttgttaaatcgtacgccttatacagttgcgccttatggtgtaaatgaatacaagaaatcgttaggaaattttcctacagaaatgcgcgaaggttttatttttgtttaccaagatgtgagaggaaaatggatgagcgaaggcgaatttgaagatgttcgacctataaatccttcaaaaagtaaaaaggcaattgacgaaagcacagatacatttgatacgctagaatggcttgctaaaaacttgaagaattacacgaaaaaagctggaatttatggaatttcgtatcctggtttttattcgacaatgagtttggttaattcgcatccaactctaaaagccgtttcgccacaagcgcccgttaccaattggtttttaggtgacgattttcatcataatggagttttattcttgaatgattctttctcatttatgactttttttggtgtaaaacgtccgcaaccaattacgccagataaaggtccgaaacgttttgaatatccaataaaagataattatagattttatgcaagtggctctgtaaaagagttgaaagataaatatttgcaagataatatcaagttttacaatgatttatttgcgcatccagattacgatcaattttggcaagatcgtaatgttttaccacatttaactaacgtgcaacctgctgtaatgacggttggaggtttttttgatgcagaagatgtctacggcgctttcgaaacgtataaagcaattgagaaacaaaatccgaaagcaacaaatattatggttgccggaccttggtttcatggtggttgggttcgtagcaacggaagtacttttggagatatgcaatttgcatcgaatacaagtgagcattatcagcaagaaatagaattgcctttttttaattattacttaaaagataaaggtaattttaaaccaaccgaagctacaatttttattacgggatctaacgaatggaaacaatttgatgcttggccaccaaaaaatgtaacaacacaaaaaatttatttgcaacaaaatggtaaaatagcttttaataaaaccaatacaacaactacttttgacgaatatgttgcagatccaaattctccagttccttattcaggaggagttttagaaactcgttcaagagaatatatggtcgatgatcaacgctttgcttctactcgtcctgatgttatggtgtatcaatctgatattttgacagaagatattacgcttgctggtcctgttatcaatcatttagtggtttctactacgggaacagacgctgattatgttgtaaaattgattgatgtttatcctgaaaacacgccaaaatttaataacaaattaatggctggatatcaaaatttgattcgtgcagaaattatgcgcggaaaatatagaaatagtttctctaaccccgaagctatggttccgaataaagaaacaaatgtaacgtacacgatgccagatgttggacatacatttaagaaaggacatcgcattatgattcaagttcagaacagttggtttcctttagcagatcgcaatccgcaacaatttatgaatgtttacgaagcaacttctaaagattatttaaaacaaacgcaacgaatttatcatacttcttatatcgaaattccggtattgaaataacaaaaaaatccagctaattagctggattttttttataatgttacttttcctatttttcctttatttccaactaaaattacatattttttatcgggcgaaaccgtacaagtatg

SEQ ID NO: 12 - protein, 616 aa, Empedobacter brevis
VKKLTLKVTLLTLLLGSTVGFAQDAKADSAYVRDNYEKIEQVIPMRDGTKLFTAIYQPKDKTKQYPVLLNRTPYTVAPYGVNEYKKSLGNFPTEMREGFIFVYQDVRGKWMSEGEFEDVRPINPSKSKKAIDESTDTFDTLEWLAKNLKNYTKKAGIYGISYPGFYSTMSLVNSHPTLKAVSPQAPVTNWFLGDDFHHNGVLFLNDSFSFMTFFGVKRPQPITPDKGPKRFEYPIKDNYRFYASGSVKELKDKYLQDNIKFYNDLFAHPDYDQFWQDRNVLPHLTNVQPAVMTVGGFFDAEDVYGAFETYKAIEKQNPKATNIMVAGPWFHGGWVRSNGSTFGDMQFASNTSEHYQQEIELPFFNYYLKDKGNFKPTEATIFITGSNEWKQFDAWPPKNVTTQKIYLQQNGKIAFNKTNTTTTFDEYVADPNSPVPYSGGVLETRSREYMVDDQRFASTRPDVMVYQSDILTEDITLAGPVINHLVVSTTGTDADYVVKLIDVYPENTPKFNNKLMAGYQNLIRAEIMRGKYRNSFSNPEAMVPNKETNVTYTMPDVGHTFKKGHRIMIQVQNSWFPLADRNPQQFMNVYEATSKDYLKQTQRIYHTSYIEIPVLK

SEQ ID NO: 13 - DNA, 40 bp, Artificial Sequence
  Synthetic DNA
gtatcacgaggccctagctgtggtgtcatggtcggtgatc

SEQ ID NO: 14 - DNA, 40 bp, Artificial Sequence
  Synthetic DNA
ttcggggattccatatgataccctttttacgtgaacttgc

SEQ ID NO: 15 - DNA, 38 bp, Artificial Sequence
  Synthetic DNA
gggaattccatatgaaaaaattaacattaaaagtaact

SEQ ID NO: 16 - DNA, 36 bp, Artificial Sequence
  Synthetic DNA
gggggctgcagtacttgtacggtttcgcccgataaa

SEQ ID NO: 17 - DNA, 36 bp, Artificial Sequence
  Synthetic DNA
cgttaacgctttcgcgcaagatgcaaaagcagattc

SEQ ID NO: 18 - DNA, 30 bp, Artificial Sequence
  Synthetic DNA
cgcctgcagcatacttgtacggtttcgccc

SEQ ID NO: 19 - DNA, 27 bp, Artificial Sequence
  Synthetic DNA
cgctctagaattttttcaatgtgattt

SEQ ID NO: 20 - DNA, 36 bp, Artificial Sequence
  Synthetic DNA
gcttttgcatcttgcgcgaaagcgttaacggaaaac

SEQ ID NO: 21 - DNA, 33 bp, Artificial Sequence
  Synthetic DNA
ggggatcccatatgaaaaagcgcgttctcgccc

SEQ ID NO: 22 - DNA, 30 bp, Artificial Sequence; identical to SEQ ID NO: 18
  Synthetic DNA

SEQ ID NO: 23 - DNA, 32 bp, Artificial Sequence
  Synthetic DNA
ggaattccatatgcccttctcccgtaccctgc

SEQ ID NO: 24 - DNA, 33 bp, Artificial Sequence
  Synthetic DNA
cgggatccctgatctttcaggattttagggttg

SEQ ID NO: 25 - DNA, 39 bp, Artificial Sequence
  Synthetic DNA
tccgttaacgctttcgcggcgccacccctgtcgatgacc

SEQ ID NO: 26 - DNA, 33 bp, Artificial Sequence
  Synthetic DNA
ttgacgtcttactgatctttcaggattttaggg

SEQ ID NO: 27 - DNA, 26 bp, Artificial Sequence
  Synthetic DNA
gctctagaattttttcaatgtgattt

SEQ ID NO: 28 - DNA, 40 bp, Artificial Sequence
  Synthetic DNA
catcgacaggggtggcgccgcgaaagcgttaacggaaaac

(19) United States
(12) Patent Application Publication
(10) Pub. No.: US 2005/0227325 A1
(43) Pub. Date: Oct. 13, 2005

(54) Recombinant polynucleotide

(75) Inventors: Yasuhiro Mihara, Kanagawa (JP); Yoshinori Hirao, Kanagawa (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 11/085,576

(22) Filed: Mar. 22, 2005

(30) Foreign Application Priority Data
Mar. 22, 2004 (JP) 2004-083481

(51) Int. Cl. C12P 21/06; C07H 21/04; C12N 1/21; C07K 14/245; C12N 15/74

(52) U.S. Cl. 435/69.1; 435/252.33; 435/488; 530/350; 536/23.7

(57) ABSTRACT

A highly expressible recombinant polynucleotide is constructed. The recombinant polynucleotide contains a nucleotide sequence encoding a signal peptide derived from the N-terminal region of acid phosphatase present in bacterium belonging to Enterobacteriaceae. In addition the recombinant polynucleotide may contain a nucleotide sequence encoding a protein integrated downstream of the nucleotide sequence encoding the signal peptide. A transformant is constructed by introducing the recombinant polynucleotide to produce the protein.

### CROSS-REFERENCE TO RELATED APPLICATIONS

The present application claims the benefit of priority based on the Japanese Patent Application No. 2004-083481 filed on Mar. 22, 2004, the entire disclosure of which is incorporated herein by reference.

### BACKGROUND OF THE INVENTION

1. Field of the Invention

The present invention relates to recombinant polynucleotides and, more specifically, to highly expressible recombinant polynucleotides. The present invention also relates to transformants and a process of protein production that make use of such recombinant polynucleotides.

2. Description of the Related Art

One of the most essential techniques used in the rapidly developing field of genetic engineering is the introduction of foreign genes into living organisms to create transformants that can express the foreign genes. In an effort to increase the expression levels of the foreign genes, various expression vectors and other recombinant constructs have been devised and used as an important tool of genetic engineering. For example, promoters and other regulatory sequences of genes have been extensively studied: new promoters have been discovered and known promoters have been modified.

It has been known that signal peptides of certain bacterial secretory proteins can be used to increase the expression levels of certain proteins whose expression levels are generally low. Techniques are known that make use of the signal peptide of OmpA of E. coli (see, for example, Non-patent Article 1) or the signal peptide of α-amylase of Bacillus subtilis (see, for example, Non-patent Article 2).

Non-patent Article 1: Ghrayeb et. al., Secretion cloning vectors in Escherrichia coli., EMBO J., 3, 2437-2442 (1984)

Non-patent Article 2: Palva I et. al., Secretion of Escherichia coli β-lactamase from Bacillus subtilis by the aid of α-amylase signal sequence., Biotechnology., 24, 344-348 (1992)

### SUMMARY OF THE INVENTION

Various types of recombinant constructs have been developed thus far to promote gene expression. In practice, however, mere combination of existing technologies cannot necessarily provide sufficiently effective recombinant constructs for gene expression and, thus, a demand exists for even more effective constructs.

Accordingly, it is an objective of the present invention to provide a highly expressible recombinant polynucleotide. It is another objective of the present invention to provide a transformant and a process for protein production that make use of the highly expressible recombinant polynucleotide.

In the course of our extensive study, the present inventors have discovered that a highly expressible recombinant polynucleotide can be obtained by the use of certain signal peptides, the discovery leading the present inventors to complete the present invention. Thus, the present invention provides the following recombinant polynucleotides:

1) A recombinant polynucleotide, comprising a nucleotide sequence that encodes a signal peptide derived from the N-terminal region of acid phosphatase present in bacterium belonging to Enterobacteriaceae.

2) The recombinant polynucleotide according to item 1, wherein the bacterium belonging to Enterobacteriaceae is selected from the group consisting of Enterobacter aerogenes, Escherichia blattae, Klebsiella planticola, Morganella morganii, Providencia stuartii, and Shigella flexneri.

3) A recombinant polypeptide, comprising a nucleotide sequence that encodes the signal peptide of SEQ ID NO: 1 in the sequence listing.

4) The recombinant polynucleotide according to any one of items 1 to 3, comprising a nucleotide sequence encoding a protein integrated downstream of the nucleotide sequence encoding the signal peptide.

5) The recombinant polynucleotide according to item 4, wherein the protein is selected from the group consisting of:
(A) a protein having the amino acid sequence of SEQ ID NO:3 in the sequence listing; and
(B) a protein having a peptide-synthesizing activity and having the amino acid sequence that includes one or several amino acids mutation in the amino acid sequence of SEQ ID NO: 3, the mutation being selected from the group consisting of substitution, deletion, insertion, addition, and inversion.

6) The recombinant polynucleotide according to item 4, wherein the protein is selected from the group consisting of:
(C) a protein having the amino acid sequence of SEQ ID NO:4 in the Sequence Listing; and
(D) a protein having an arginine racemase activity and having the amino acid sequence that includes one or several amino acids mutation in the amino acid sequence of SEQ ID NO: 4, the mutation being selected from the group consisting of substitution, deletion, insertion, addition, and inversion.

7) A transformant, comprising a recombinant polynucleotide that contains a nucleotide sequence encoding a signal peptide derived from the N-terminal region of acid phosphatase present in bacterium belonging to Enterobacteriaceae and contains a nucleotide sequence encoding a protein integrated downstream of the nucleotide sequence encoding the signal peptide.

8) A transformant according to item 7, wherein the bacterium belonging to Enterobacteriaceae is selected from the group consisting of Enterobacter aerogenes, Escherichia blattae, Klebsiella planticola, Morganella morganii, Providencia stuartii, and Shigella flexneri.

9) A transformant according to item 7,
wherein the protein is selected from the group consisting of:
(A) a protein having the amino acid sequence of SEQ ID NO:3 in the sequence listing; and
(B) a protein having a peptide-synthesizing activity and having the amino acid sequence that includes one or several amino acids mutation in the amino acid sequence of SEQ ID NO: 3, the mutation being selected from the group consisting of substitution, deletion, insertion, addition, and inversion.

10) A transformant according to item 7,
wherein the protein is selected from the group consisting of:
(C) a protein having the amino acid sequence of SEQ ID NO:4 in the Sequence Listing; and
(D) a protein having an arginine racemase activity and having the amino acid sequence that includes one or several amino acids mutation in the amino acid sequence of SEQ ID NO: 4, the mutation being selected from the group consisting of substitution, deletion, insertion, addition, and inversion.

11) A transformant, comprising a recombinant polynucleotide that contains a nucleotide sequence encoding a signal peptide of SEQ ID NO: 1 in the sequence listing and contains a nucleotide sequence encoding a protein integrated downstream of the nucleotide sequence encoding the signal peptide.

12) A transformant according to item 11,
wherein the protein is selected from the group consisting of:
(A) a protein having the amino acid sequence of SEQ ID NO:3 in the sequence listing; and
(B) a protein having a peptide-synthesizing activity and having the amino acid sequence that includes one or several amino acids mutation in the amino acid sequence of SEQ ID NO: 3, the mutation being selected from the group consisting of substitution, deletion, insertion, addition, and inversion.

13) A transformant according to item 11,
wherein the protein is selected from the group consisting of:
(C) a protein having the amino acid sequence of SEQ ID NO:4 in the Sequence Listing; and
(D) a protein having an arginine racemase activity and having the amino acid sequence that includes one or several amino acids mutation in the amino acid sequence of SEQ ID NO: 4, the mutation being selected from the group consisting of substitution, deletion, insertion, addition, and inversion.

14) A production method of a protein, wherein the transformant of item 7 is cultivated in a growth medium, so that the protein accumulates in the growth medium and/or in the microbial cells.

15) A production method of a protein, wherein the transformant of item 8 is cultivated in a growth medium, so that the protein accumulates in the growth medium and/or in the microbial cells.

16) A production method of a protein, wherein the transformant of item 9 is cultivated in a growth medium, so that the protein accumulates in the growth medium and/or in the microbial cells.

17) A production method of a protein, wherein the transformant of item 10 is cultivated in a growth medium, so that the protein accumulates in the growth medium and/or in the microbial cells.

18) A production method of a protein, wherein the transformant of item 11 is cultivated in a growth medium, so that the protein accumulates in the growth medium and/or in the microbial cells.

19) A production method of a protein, wherein the transformant of item 12 is cultivated in a growth medium, so that the protein accumulates in the growth medium and/or in the microbial cells.

20) A production method of a protein, wherein the transformant of item 13 is cultivated in a growth medium, so that the protein accumulates in the growth medium and/or in the microbial cells.

Thus, the present invention provides highly expressible recombinant polynucleotides and transformants. The present invention also provides an effective process of protein production.

The present invention provides a valuable tool for use in the field of biotechnology and offers an effective genetic-engineering technique suitable for the protein production.

### DETAILED DESCRIPTION OF THE PREFERRED EMBODIMENTS

Several embodiments of the present invention will now be described including the best mode of carrying out the invention.

It may be appreciated that various textbooks and laboratory manuals are available describing standard techniques of genetic engineering and all of the procedures described below will be readily implemented by anyone skilled in the art by referring to these literatures. Among such literatures are Molecular cloning, 2nd edition, Cold Spring Harbor Press (1989); A Hand Book of Cell Engineering, Toshio Kuroki et al., Yodo-sha (1992); and A New Hand Book of Genetic Engineering 3rd ed., Muramatsu et al., Yodo-sha (1999).

1. Recombinant Polynucleotide and Transformant of the Invention

A recombinant polynucleotide of the present invention is a recombinant construct having a nucleotide sequence coding for a certain signal peptide. The signal peptide may be a signal peptide derived from the N-terminus of acid phosphatase of bacteria of enterobacteriaceae. More specifically, the signal peptide for use in the present invention may be a signal peptide present in a precursor protein of acid phosphatase of enterobacteriaceae.

Signal peptides are about 10 to 50 amino acid-long, preferably about 15 to 30 amino acid-long peptides derived from the N-terminus of immature precursor proteins. In general, signal peptides are considered to be responsible for the translocation of secretory proteins across the membrane.

The signal peptide for use in the present invention was originally isolated from the precursor protein of acid phosphatase of enterobacteria. Examples of such enterobacteria may include microorganisms belonging to genus Enterobacter, genus Escherichia, genus Klebsiella, genus Morganella, genus Providencia or genus Shigella. More specific examples of such enterobacteria may include Enterrobacter aerogenes, Escherichia blattae, Klebsiella planticola, Morganella morganii, Providencia stuartii, and Shegella flexneri.

One example of the signal peptide for use in the present invention is the signal peptide having the amino acid sequence described in the sequence listing as SEQ ID NO: 1. When the signal peptide with the amino acid sequence of SEQ ID NO: 1 is used in the recombinant polynucleotide of the present invention, the polynucleotide incorporates a nucleotide sequence encoding the signal peptide of SEQ ID NO: 1 (hereinafter, SEQ ID NOs are as defined in the sequence listing unless otherwise specified) The nucleotide sequence encoding the signal peptide of SEQ ID NO: 1 in accordance with the present invention may be any of the plurality of nucleotide sequences that are translated into the amino acid sequence of SEQ ID NO: 1 (according to the codon table). One such nucleotide sequence encoding the amino acid sequence of SEQ ID NO: 1 is described in SEQ ID NO: 2.

The term “polynucleotide” as used herein encompasses DNA, RNA, hybrids, and chimeras thereof. The polynucleotide may be single-stranded or double-stranded. The recombinant polynucleotide of the present invention is preferably DNA because of its stability and easy handling.

Another preferred embodiment of the recombinant polynucleotide of the present invention is a recombinant polynucleotide in which a nucleotide sequence encoding a desired protein is integrated downstream of the nucleotide sequence encoding the signal peptide. The recombinant polynucleotide of the present invention is capable of high-level expression of the protein downstream of the nucleotide sequence of the signal peptide, which makes the recombinant polynucleotide suitable for use as an expression vector.

While the vector for use in the present invention may be any suitable vector, common vectors, such as plasmids and viruses, are preferably used. Examples of such vectors may include pUC19, pUC18, pBR322, pHSG299, pHSG298, pHSG399, pHSG398, RSF1010, pMW119, pMW118, pMW219, and pMW218. Phage DNAs are another preferred vector.

The recombinant polynucleotide of the present invention may further incorporate other nucleotide sequences commonly used in expression vectors. For example, the polynucleotide of the present invention may incorporate a promoter, terminator, Kozak's sequence, multicloning site, drug-resistant gene for selecting transformants, and other nucleotide sequences. The promoter for use in the present invention may be any promoter commonly used in the production of heterologous proteins in E. coli. Examples may include T7 promoter, lac promoter, trp promoter, trc promoter, tac promoter, PR promoter of lambda phage, PL promoter, and other strong promoters. Examples of the terminator may include T7 terminator, fd phage terminator, T4 terminator, terminator of tetracycline-resistant gene, and E. coli trp A gene terminator.

Examples of proteins to be expressed may include the following proteins (A) through (D):
(A) a protein having the amino acid sequence of SEQ ID NO: 3 in the sequence listing;
(B) a protein having a peptide-synthesizing activity and having the amino acid sequence that includes one or several amino acids mutation in the amino acid sequence of SEQ ID NO: 3, the mutation being selected from the group consisting of substitution, deletion, insertion, addition, and inversion;
(C) a protein having the amino acid sequence of SEQ ID NO: 4 in the sequence listing; and
(D) a protein having an arginine racemase activity and having the amino acid sequence that includes one or several amino acids mutation in the amino acid sequence of SEQ ID NO: 4, the mutation being selected from the group consisting of substitution, deletion, insertion, addition, and inversion.

The term “peptide-synthesizing activity” as used herein refers to an activity that promotes the reaction in which peptide bonds are formed between two or more compounds to generate a new compound having peptide bonds. More specifically, the term refers to an activity that promotes the reaction between two amino acids or esters thereof to form peptide bonds and thereby generate a peptide compound with the number of peptide bonds increased by at least one. The term “arginine racemase activity” as used herein refers to an activity that promotes the reaction in which arginine is racemized.

As used herein, the term “several” refers to any number that does not significantly affect the conformation and activity of the resulting protein if that number of amino acid changes have occurred in the protein, although the number may vary depending on the positions of the amino acid residues in the protein conformation and the type of the amino acid residues. Specifically, the number is in the range of 2 to 50, preferably 2 to 30, and more preferably 2 to 10. When the protein to be expressed is the protein (B) or the protein (D) with its amino acid sequence containing substitution, deletion, insertion, addition, and/or inversion of one or several amino acid residues, it is desirable that the protein retain, at 50° C. and at pH 8, preferably half or more, more preferably 80% or more, even more preferably 90% or more, and still more preferably 95% or more of its enzymatic activity of the protein containing no mutation. For example, in the case of the protein (B), which results from substitution, deletion, insertion, addition, and/or inversion of one or several amino acid residues in the amino acid sequence of SEQ ID NO: 2, it is desirable that the protein retain, at 50° C. and at pH 8, preferably half or more, more preferably 80% or more, even more preferably 90% or more, and still more preferably 95% or more of its enzymatic activity of the protein having the amino acid sequence of SEQ ID NO: 2.

The mutation in amino acids as shown in the aforementioned (B) and (D) may be induced by modifying the nucleotide sequence so that the amino acids at the corresponding sites of the enzyme gene may be substituted, deleted, inserted, and/or added using, for example, the site-specific mutation method. Alternatively, the polynucleotide having modified nucleotide sequence may be obtained by the conventionally known the mutation process. The mutation process may include a technique involving a step for in vitro inducing the mutation in the DNA encoding the amino acids of the (A) or (C) under the treatment of hydroxylamine and a technique involving a step for introducing the mutation in a bacterium belonging to the genus Escherichia containing the DNA encoding the amino acids of the (A) or (C) group by UV irradiation, or under the treatment with methyl-N′-nitro-N-nitrosoguanidine (NTG) or any of mutating agents generally used for mutation engineering, such as nitrous acid.

The mutations such as the aforementioned substitution, deletion, insertion, addition and/or inversion in nucleotides include inherent mutation induced by a difference among the species of microorganisms or the strains and mutation induced naturally. The DNA incorporating such variation is introduced in proper cells for expression. Subsequently, the expressed product is examined for the enzymatic activity. In this manner, DNA can be obtained that encodes a substantially identical protein to the protein of SEQ ID NO: 2 or SEQ ID NO: 3.

The protein with the amino acid sequence of SEQ ID NO: 2 was isolated from Empedobacter brevis (strain FERM BP-8113; Depository institution, International Patent Organism Depository (IPOD), National Institute of Advanced Industrial Science and Technology (AIST); Address, 1-1, Chuo 6, Higashi 1-chome, Tsukuba-shi, Ibaraki-ken, Japan; Date of International Deposition, Jul. 8, 2002). The amino acid sequence described in SEQ ID NO: 2 is the mature protein region having the amino acid sequence described in SEQ ID NO: 12.

The amino acid sequence of SEQ ID NO: 12 is encoded by, for example, the nucleotide sequence described in SEQ ID NO: 11. The DNA consisting of bases numbers from 61 to 1908 described in SEQ ID NO: 11 corresponds to the coding sequence (CDS). The nucleotide sequence consisting of bases numbers from 61 to 1908 includes the region encoding the signal peptide and the region encoding the mature protein. The region from base number 61 to 126 encodes the signal peptide and the region from base number 127 to 1908 encodes the mature protein. The signal peptide encoded by the sequence from base number 61 to 126 originally exists in the above-mentioned bacteria. The main function of the signal peptide is inferred to play an important role in translocation of certain proteins across the cell membrane. The region from base number 127 to 1908 encodes the mature protein, which has the amino acid sequence of SEQ ID NO: 2 and has high peptide-synthesizing activity.

The protein having the amino acid sequence of SEQ ID NO: 3 can be isolated from, for example, Pseudomonas taetrolents (strain ATCC 4683). The cells referenced by an ATCC accession number are deposited at American Type Culture Collection (ATCC) (P.O. Box 1549 Manassas, Va. 20110, US) and can be obtained from the institution by referring to this number.

It is a common practice to isolate a polynucleotide having the nucleotide sequence of a protein of interest from microorganisms expressing the protein and insert the polynucleotide into a recombinant construct, such as a vector.

2. Transformed Microorganisms of the Present Invention

The transformed microorganisms of the present invention are those into which the above-described recombinant polynucleotide of the present invention has been introduced.

Hosts for expressing the protein of interest may be Escherichia coli and other bacteria belonging to genus Escherichia.

Among conventional transformation techniques for introducing a recombinant DNA into host cells are a technique by D. M. Morrison (Methods in Enzymology 68, 326 (1979)) and a technique in which the recipient cells are treated with calcium chloride to increase the permeability to DNA (Mandel, M. and Higa, A., J. Mol. Biol., 53, 159 (1970)).

3. Method for Protein Production According to the Present Invention

The protein production method of the present invention uses the above-described transformed microorganism to produce a protein of interest. Specifically, the method of the present invention involves cultivating the transformed microorganism in a culture medium so that the protein of interest accumulates in the culture medium and/or in the cells. The accumulated protein may be recovered and purified by using a known technique.

The cells of the microorganism can be obtained by cultivating the microorganism in a proper culture medium that is appropriate for the type of the microorganism used. This culture medium may be any culture medium suitable for the growth of the microorganism and may be a common culture medium containing a carbon source, nitrogen source, phosphorus source, sulfur source, inorganic ions, and, if necessary, organic nutrients.

The carbon source may be any carbon source that can be utilized by the microorganism. Examples may include sugars, such as glucose, fructose, maltose, and amylose; alcohols, such as sorbitol, ethanol, and glycerol; organic acids, such as fumaric acid, citric acid, acetic acid and propionic acid, and salts thereof; hydrocarbons, such as paraffin; and mixtures thereof.

Examples of the nitrogen source may include inorganic ammonium salts, such as ammonium sulfate and ammonium chloride; ammonium salts of organic acids, such as ammonium fumarate and ammonium citrate; nitrates such as sodium nitrate and potassium nitrate; organic nitrogen compounds, such as peptone, yeast extracts, broths, and corn steep liquor; and mixtures thereof.

If necessary, inorganic salts, trace metal salts, vitamins, and other nutrients commonly used in a culture medium may be added.

Conditions for cultivating the microorganism are not limited to particular conditions, either. For example, the cells may be cultivated aerobically for 12 to 48 hours while the pH and the temperature are properly controlled in the ranges of from pH 5 to 8 and from about 15 to 40° C., respectively.

Other conditions for cultivating the cells and inducing protein production are properly selected depending on the type of the marker for the vector used, promoter, and the host bacteria.

When a large-scale production of a protein using a recombinant DNA technique is desired, the protein may form an inclusion body in the cells of the transformant. This may be utilized in one preferred embodiment of the present invention. The advantages of this approach are that the desired protein is protected from digestion by proteases present in the cells, and that the desired protein can be easily purified by disrupting the cells, followed by centrifugation.

### EXAMPLES

### Example 1

### Modification of Promoter Sequence of Acid Phosphatase Gene Derived from Enterobacter aerogenes

According to the description in Journal of Bioscience and Bioengineering (2001) Vol. 92, No. 1, 50-54, a 1.6 kbp DNA fragment containing an acid phosphatase gene was excised from chromosomal DNA of Enterobacter aerogenes strain IFO 12012 with restriction enzymes Sal I and Kpn I and was isolated. The fragment was ligated to pUC118 to construct a plasmid pEAP120. pEAP120 is a plasmid containing a promoter of acid phosphatase gene and a nucleotide sequence encoding a signal peptide. It should be noted that microorganisms referenced by IFO numbers were originally deposited at Institution for Fermentation, Osaka (17-85, Juso-honmachi 2-chome, Yodogawa-ku, Osaka 532-8686, Japan) but were later transferred to National Institute of Technology and Evaluation (NITE), Department of Biotechnology, NITE Biological Resource Center as the work of IFO was transferred to NITE on Jun. 30, 2002. Thus, the microorganisms can now be obtained from NBRC by referring to IFO numbers.

Subsequently, the promoter sequence upstream of the gene was partially modified to enhance its activity. Specifically, site-specific mutagenesis was performed using Quikchange Site-Directed Mutageneis Kit (supplied from STRATAGENE) to substitute the −10 region of the putative promoter sequence of the acid phosphatase gene from AAAAAT to TATAAT. Meanwhile, PCR oligonucleotide primers EM1 (5′-CTT ACA GAT GAC TAT AAT GTG ACT AAA AAC; SEQ ID NO: 5) and EMR1 (5′-GTT TTT AGT CAC ATT ATA GTC ATC TGT AAG; SEQ ID NO: 6), designed for mutagenesis, were synthesized. According to the instruction, mutations were introduced using pEAP120 as a template. By using the Dye Terminator technique with DNA sequencing kit Dye Terminator Cycle Sequencing Ready Reaction (supplied from PERKIN ELMER), the nucleotide sequence was determined on a 310 Genetic analyzer (supplied from ABI) to confirm that the desired mutations had been introduced. The plasmid was named pEAP130. pEAP130 is a plasmid containing a nucleotide sequence encoding the signal peptide derived from N-terminus of acid phosphatase and the modified promoter.

E. coli strain JM109 was transformed with each of the plasmids to make transformants E. coli JM109/pEAP120 and E. coli JM109/pEAP130. Each transformant was inoculated onto to a 50 ml LB medium (10 g/l tryptone, 5 g/l yeast extract, 10 g/l sodium chloride, pH 7.0) containing 100 mg/l ampicillin in a 500 ml Sakaguchi flask and was cultivated at 37° C. for 16 hours. 1 mM IPTG, when used, was added at the beginning of the cultivating period.

After the cultivating period, the culture solution was centrifuged to collect the cells. The cells were then washed once in a saline, were suspended in a 5 ml 100 mM potassium phosphate buffer (pH7.0), and were then sonicated at 4° C. for 20 minutes. The sonicated suspension was centrifuged to remove insoluble fractions and the supernatant was recovered as a crude enzyme extract.

Using a protein assay kit (supplied from Bio-Rad) and BSA as a standard protein, the protein concentration in the crude enzyme extract was determined according to the Bradford technique. Since the acid phosphatase, other than its phosphatase activity, exhibits a phosphotransferase activity on nucleoside (Journal of Bioscience and Bioengineering (2001) Vol. 92, No. 1, 50-54). This activity was determined in the following manner as a measure of the expression level of the protein and a comparison was made. A reaction mixture (1 ml) was prepared containing 40 μmol/ml inosine, 100 μmol/ml sodium pyrophosphate, 100 μmol/ml sodium acetate buffer (pH5.0) and the enzyme, and the reaction was allowed to proceed at pH 5.0, 30° C., for 10 minutes. Subsequently, a 200 μl 2N hydrochloric acid was added to terminate the reaction. The resultant mixture was then centrifuged and the precipitate was removed. The amount of 5′-inosinic acid produced by the phosphotransfer reaction was determined: an enzyme level capable of producing 1 μmol 5′-inosinic acid every one minute under the specified reaction conditions was defined as 1 unit.

Inosine and 5′-inosinic acid were analyzed by HPLC(High-performance liquid chromatography) under the following condition: column=Cosmosil 5C18-AR (4.6×150 mm) (supplied from Nacalai Tesque), mobile phase=5 mM potassium phosphate buffer (pH 2.8)/methanol=95/5, flow rate 1.0 ml/min, temperature=room temperature, and UV detection made at 245 nm.

The activity of the cell-free extracts prepared from E. coli strain JM109 transformed with the respective plasmids was determined and the results are shown in Table 1 below. As shown Table 1, E. coli JM109/pEAP120 showed significantly low activity in the absence of IPTG, whereas E. coli JM109/pEAP130 in the absence of IPTG showed high activity comparable to the activity of E. coli JM109/pEAP120 observed in the presence of IPTG. This demonstrates that the modification of the promoter in the −10 region is effective. In the following examples, expression plasmids are constructed that are under control of the modified promoter, which enables high expression level in the absence of IPTG, and in which a heterologous gene is inserted downstream of the sequence of signal peptide (signal sequence).

TABLE 1

| | | Activity |
|---|---|---|
| Strains | Culture | (units/mg) |
| Escherichia coli | IPTG added | 0.357 |
| JM109/pEAP120 | | |
| Escherichia coli | IPTG free | 0.059 |
| JM109/pEAP120 | | |
| Escherichia coli | IPTG free | 0.320 |
| JM109/pEAP130 | | |

### Example 2

### Construction of Plasmids Capable of High Level Expression of Peptide-Synthesizing Enzyme Gene Derived from Genus Empedobacter

### Preparation Example 1

### Isolation of Peptide-Synthesizing Enzyme Gene from Empedobacter brevis

Hereinafter, isolation of a gene for peptide-synthesizing enzyme will be explained. Empedobacter brevis strain FERM BP-8113 was used as the microorganism. For isolating the gene, Escherichia coli JM-109 was used as a host while pUC118 was used as a vector.

(1) Preparation of PCR Primer Based on Determined Internal Amino Acid Sequence

A mixed primer having the nucleotide sequences indicated in SEQ ID NO: 9 and SEQ ID NO: 10, respectively, was constructed based on the amino acid sequences (SEQ ID NO: 7 and 8) determined by the Edman's decomposition method detecting the digestion product of lysyl endopeptidase of a peptide-synthesizing enzyme derived from the Empedobacter brevis strain FERM BP-8113.

(2) Preparation of Microbial Cells

Empedobacter brevis strain FERM BP-8113 was cultivated at 30° C. for 24 hours on a CM2G agar medium (containing glucose 50 g/l, yeast extract 10 g/l, peptone 10 g/l, sodium chloride 5 g/l, and agar 20 g/l, pH 7.0). One loopful cells of the resulting microbial cells was inoculated into a 500 ml Sakaguchi flask containing 50 ml of a CM2G liquid medium (the aforementioned medium excluding agar) followed by shake culture at 30° C.

(3) Preparation of Chromosomal DNA from Microbial Cells

50 ml of culture solution was centrifuged (12,000 rpm, 4° C., 15 minutes) to recover the microbial cells. Then, a chromosomal DNA was obtained from the microbial cells using the QIAGEN Genomic-Tip System (supplied from Qiagen) based on the procedure described in the manual therefor.

(4) Preparation of DNA Fragment Containing Part of Gene for Peptide-Synthesizing Enzyme by PCR

A DNA fragment containing a portion of the gene for the peptide-synthesizing enzyme derived from Empedobacter brevis strain FERM BP-8113 was obtained by the PCR method using LA-Taq (supplied from Takara Shuzo). A PCR reaction was then carried out by using the primers having the nucleotide sequences of SEQ ID NOs: 9 and 10 to a chromosomal DNA as template obtained from Empedobacter brevis strain FERM BP-8113.

The PCR reaction was carried out for 30 cycles under the following conditions using the Takara PCR Thermal Cycler PERSONAL (supplied from Takara Shuzo).

| | |
|---|---|
| 94° C. | 30 seconds |
| 52° C. | 1 minute |
| 72° C. | 1 minute |

After completion of the reaction, 3 ml of the reaction mixture was applied to 0.8% agarose electrophoresis. As a result, it was verified that a DNA fragment of about 1.5 kilobases (kb) was amplified.

(5) Cloning of Gene for Peptide-Synthesizing Enzyme from Gene Library

In order to obtain the gene for peptide-synthesizing enzyme in full-length, southern hybridization was carried out by using the DNA fragment amplified in the PCR procedure as a probe. The procedure for southern hybridization is explained in Molecular Cloning, 2nd edition, Cold Spring Harbor Press (1989).

Specifically, the approximately 1.5 kb DNA fragment amplified by the PCR procedure was separated by 0.8% agarose electrophoresis. The target band was then cut out and purified. The DNA fragment was labeled with digoxinigen as probe by using DIG High Prime (supplied from Boehringer-Mannheim) according to the procedure described in the manual therefor.

After completely digesting the chromosomal DNA of Empedobacter brevis obtained in the step (3) of the present Preparation Example 1 by reacting at 37° C. for 16 hours with restriction enzyme Hind III, the resultant was electrophoresed with on 0.8% agarose gel. After the electrophoresis, the electrophoresed chromosomal DNA was blotted onto a positively charged Nylon membrane filter (supplied from Roche Diagnostics) from the agarose gel followed by treatments consisting of alkaline denaturation, neutralization and immobilization. Hybridization was carried out by using EASY HYB (supplied from Boehringer-Mannheim). After pre-hybridizing the filter at 50° C. for 1 hour, the probe labeled with digoxinigen prepared as described above was added and hybridization was carried out at 50° C. for 16 hours. Subsequently, the filter was washed for 20 minutes at room temperature with 2×SSC containing 0.1% SDS. Moreover, the filter was additionally washed twice at 65° C. for 15 minutes with 0.1×SSC containing 0.1% SDS.

Detection of bands that hybridized with the probe was carried out by using the DIG Nucleotide Detection Kit (supplied from Boehringer-Mannheim) according to the procedure described in the manual therefor. As a result, a roughly 4 kb band was able to be detected that hybridized with the probe.

Then, 5 mg of the chromosomal DNA prepared in the step (3) of the present Preparation Example 1 was completely digested with Hind III. A roughly 4 kb of DNA was separated by 0.8% agarose gel electrophoresis, followed by purifying the DNA using the Gene Clean II Kit (supplied from Funakoshi) and dissolving the DNA in 10 ml of TE (Tris-EDTA). 4 ml of this product was then mixed with pUC118 Hind III/BAP (supplied from Takara Shuzo) and a ligation reaction was carried out by using the DNA Ligation Kit Ver. 2 (supplied from Takara Shuzo). 5 μl of the ligation reaction mixture and 100 μl of competent cells of Escherichia coli JM109 (supplied from Toyobo) were mixed to transform the Escherichia coli. This was then applied to a suitable solid medium to construct a chromosomal DNA library.

To obtain the full-length of gene for peptide-synthesizing enzyme, the chromosomal DNA library was screened by colony hybridization using the aforementioned probe. The procedure for colony hybridization is explained in Molecular Cloning, 2nd edition, Cold Spring Harbor Press (1989).

The colonies of the chromosomal DNA library were transferred on a nylon membrane filter, Nylon Membrane for Colony and Plaque Hybridization (supplied from Roche Diagnostics), followed by treatments consisting of alkali denaturation, neutralization and immobilization. Hybridization was carried out using EASY HYB (supplied from Boehringer-Mannheim). After pre-hybridizing the filter at 37° C. for 1 hour, the aforementioned probe labeled with digoxinigen was added, followed by hybridization at 50° C. for 16 hours. In addition, the filter was washed for 20 minutes at room temperature with 2×SSC containing 0.1% SDS. Moreover, the filter was additionally washed twice at 65° C. for 15 minutes with 0.1×SSC containing 0.1% SDS.

Detection of colonies hybridizing with the labeled probe was carried out by using the DIG Nucleotide Detection Kit (supplied from Boehringer-Mannheim) according to the explanation described in the manual therefor using the DIG Nucleotide Detection Kit (supplied from Boehringer-Mannheim). As a result, two strains of colonies were verified to hybridize with the labeled probe.

(6) Nucleotide Sequence of Gene for Peptide-Synthesizing Enzyme Derived from Empedobacter brevis

Plasmids possessed by Escherichia coli JM109 were prepared from the aforementioned two strains of microbial cells that were verified to hybridize with the labeled probe by using the Wizard Plus Minipreps DNA Purification System (supplied from Promega) and the nucleotide sequence of a portion where hybridization with the probe occurred and nearby was determined. The sequencing reaction was carried out by using the CEQ DTCS-Quick Start Kit (supplied from Beckman-Coulter) according to the procedure described in the manual therefor. In addition, electrophoresis was carried out by using the CEQ 2000-XL (supplied from Beckman-Coulter).

As a result, it was verified that an open reading frame that encodes a protein containing the internal amino acid sequences of the peptide-synthesizing enzyme (SEQ ID NOs: 7 and 8) did exist, thereby confirming that the open reading frame was a gene encoding the peptide-synthesizing enzyme. The nucleotide sequence of the full-length of the gene for peptide-synthesizing enzyme along with the corresponding amino acid sequence is described in SEQ ID NO: 11. As a result of analysis on the homology of the resulting open reading frame with the BLASTP program, homology was discovered between the two enzymes; it showed with a homology of 34% as at the amino acid sequence level exhibited with the α-amino acid ester hydrolase of Acetobacter pasteurianus (see Appl. Environ. Microbiol., 68(1), 211-218 (2002)), and a homology of 26% at the amino acid sequence level exhibited with the glutaryl-7ACA acylase of Brevibacillus laterosporum (see J. Bacteriol., 173(24), 7848-7855 (1991)).

(7) Expression of Gene for Peptide-Synthesizing Enzyme Derived from Empedobacter brevis in Escherichia coli

The promoter region of the trp operon on the chromosomal DNA of Escherichia coli W3110 was amplified by PCR using the oligonucleotides indicated in SEQ ID NOs: 13 and 14 as primers, and the resulting DNA fragments were ligated to a pGEM-Teasy vector (supplied from Promega). E. coli JM109 was then transformed with this resultant ligation solution, and those strains having the target plasmid in which the direction of the inserted trp promoter is inserted in the opposite to the orientation of the lac promoter were selected from ampicillin-resistant strains. Next, a DNA fragment containing the trp promoter obtained by treating this plasmid with EcoO109 I/EcoR I was ligated to an EcoO109 I/EcoR I treatment product of pU190(supplied from Takara). Escherichia coli JM109 was then transformed with this resultant ligation solution and those strains having the target plasmid were selected from ampicillin-resistant strains. Next, a DNA fragment obtained by treating this plasmid with Hind III/Pvu II was ligated with to a DNA fragment containing an rrnB terminator obtained by treating pKK223-3 (supplied from Amersham Pharmacia) with Hind III/Hinc II. E. coli JM109 was then transformed with this ligation solution, strains having the target plasmid were selected from ampicillin-resistant strains, and the plasmid was designated as pTrpT.

The target gene was amplified by PCR using the chromosomal DNA of Empedobacter brevis strain FERM BP-8113 as a template and the oligonucleotides indicated in SEQ ID NO: 15 and 16 as primers. This DNA fragment was then treated with Nde I/Pst I, and the resulting DNA fragment was ligated with the Nde I/Pst I treatment product of pTrpT. Escherichia coli JM109 was then transformed with this ligation solution, those strains having the target plasmid were selected from ampicillin-resistant strains, and this plasmid was designated as pTrpT_Gtg2.

Escherichia coli JM109 having pTrpT_Gtg2 was pre-cultivated at 30° C. for 24 hours in LB medium containing 100 mg/l of ampicillin. 1 ml of the resulting culture solution was transferred in a 500 ml Sakaguchi flask containing 50 ml of a medium (D-glucose 2 g/l, yeast extract 10 g/l, casamino acids 10 g/l, ammonium sulfate 5 g/l, potassium dihydrogen phosphate 3 g/l, dipotassium hydrogen phosphate 1 g/l, magnesium sulfate heptahydrate 0.5 g/l, and ampicillin 100 mg/l), followed by cultivation at 25° C. for 24 hours. The culture solution had an α-L-aspartyl-phenylalanine-β-methyl ester forming activity of 0.11 Upper 1 ml of culture solution and it was verified that the cloned gene was expressed by E. coli. Furthermore, no activity was detected for a transformant in which only pTrpT had been introduced as a control.

(1) Construction of Plasmids Capable of High Level Expression of Peptide-Synthesizing Enzyme Derived from Genus Empedobacter

Using PCR technique, an expression plasmid was constructed that includes the gene encoding mature protein of peptide-synthesizing enzyme integrated downstream of the modified promoter and the signal sequence of acid phosphatase derived from Enterobacter aerogenes.

Specifically, the pTrpT_Gtg2 plasmid of Preparation Example 1 as a template, 0.4 mM each of oligonucleotides CAP1 (5′-CGT TAA CGC TTT CGC GCA AGA TGC AAA AGC AGA TTC: SEQ ID NO: 17) and CAP2 (5′-CGC CTG CAG CAT ACT TGT ACG GTT TCG CCC: SEQ ID NO: 18) as primers, a buffer for KOD plus (supplied from Toyobo), 0.2 mM each of dATP, dCTP, dGTP and dTTP, 1 mM magnesium sulfate, and 1 unit of KOD plus polymerase (supplied from Toyobo) were mixed together to form a 50 μl reaction mixture. The gene encoding mature protein of the peptide-synthesizing enzyme was amplified by performing PCR under the following conditions: an initial heat treatment at 94° C. for 30 sec, followed by 25 cycles of 94° C. for 15 sec, 55° C. for 30 sec, and 68° C. for 2 min and 30 sec. Meanwhile, using pEAP130 plasmid of Example 1 as a plasmid, along with the oligonucleotides of CAP3 (5′-CGC TCT AGA ATT TTT TCA ATG TGA TTT: SEQ ID NO: 19) and CAP4 (5′-GCT TTT GCA TCT TGC GCG AAA GCG TTA ACG GAA AAC: SEQ ID NO: 20) as primers, promoter and the signal sequence region of acid phosphatase were PCR-amplified under the same conditions as above. The reaction mixture was subjected to an agarose gel electrophoresis and the amplified DNA fragments were recovered with a Microspin column (supplied from Amersham Pharmacia Biotech).

Using a mixture of the amplified fragments as a template, along with the CAP2 and CAP3 oligonucleotides as primers, PCR was performed in a similar reaction mixture by 25 cycles of 94° C. for 15 sec, 55° C. for 30 sec, and 68° C. for 2 min and 30 sec. This gave a chimeric enzyme gene. The amplified DNA fragments were recovered with a Microspin column (supplied from Amersham Pharmacia Biotech) and were digested with Xba I and Pst I. The digested fragment was ligated to the Xba I-Pst I site of pUC19 plasmid. By using the Dye Terminator technique with DNA sequencing kit Dye Terminator Cycle Sequencing Ready Reaction (supplied from PERKIN ELMER), the nucleotide sequence of the plasmid was determined by a 310 Genetic analyzer (supplied from ABI), which confirmed that the desired recombinant gene had been obtained. The plasmid was designated as pAF110.

The pAF 110 plasmid uses the acid phosphatase promoter to express the peptide-synthesizing enzyme whereas the pTrpT_Gtg2 uses the trp promoter to express the same protein. To assess the effect of the signal peptide substitution, an expression vector was constructed in which the promoter of the pAF110 plasmid had been substituted with the trp promoter. Specifically, the pAF110 plasmid as a template, 0.4 mM each of oligopeptides, CAP5 (5′-GGG GAT CCC ATA TGA AAA AGC GCG TTC TCG CCC: SEQ ID NO: 21) and CAP6 (5′-CGC CTG CAG CAT ACT TGT ACG GTT TCG CCC: SEQ ID NO: 22) as primers, a buffer for KOD plus (supplied from Toyobo), 0.2 mM each of dATP, dCTP, dGTP and dTTP, 1 mM magnesium sulfate, and 1 unit of KOD plus polymerase (supplied from Toyobo) were mixed together to form a 50 μl reaction mixture. The chimeric gene without the promoter sequence was amplified by performing PCR under the following conditions: an initial heat treatment at 94° C. for 30 sec, followed by 25 cycles of 94° C. for 15 sec, 55° C. for 30 sec, and 68° C. for 2 min and 30 sec. The amplified DNA fragments were recovered with a Microspin column (Amersham Pharmacia Biotech) and were digested with Nde I and Pst I. The digested fragment was ligated to the Nde I-Pst I site of pTrpT plasmid of Preparation Example 1. The resulting plasmid was designated as pAF150.

(2) Assessment of the Activity of Peptide-Synthesizing Enzyme in Transformants

E. coli strain JM109 was individually transformed with pTrpT_Gtg2 of Preparation Example 1, the aforementioned plasmids pAF110 and pAF150. Each transformant was inoculated in a 3 mL LB liquid medium (10 g/l bacto tryptone, 5 g/l bacto yeast extract, and 10 g/l NaCl) containing 100 mg/l ampicillin and a pre-cultivation was carried out at 22° C. for 8 hours. Subsequently, a 2.5 ml portion resultant of the pre-cultivation was inoculated in a 50 ml Aet expression medium (10 g/l casamino acid, 10 g/l bacto yeast extract, 5 g/l (NH₄)₂SO₄, 3 g/l KH₂PO₄, 1 g/l K₂HPO₄, 2 g/l glucose, and 0.5 g/l MgSO₄.7H₂O) containing 100 mg/l ampicillin and placed in a 500 ml Sakaguchi flask. Then, shake-culture was performed at 22° C. for 20 hours to cultivate the cells. After the culturing period, the cells were recovered, were washed, and were assessed for the activity to produce alanyl-glutamine.

The activity of the cells to produce alanyl-glutamine was determined in the following manner. A reaction mixture (1 ml) containing 100 μmol/ml alanine-o-methylester (supplied from Kokusan-kagaku), 200 μmol/ml glutamine, 100 μmol/ml boric acid/sodium hydroxide buffer (pH9.0), and the cells was prepared, and the reaction was allowed to proceed at pH9.0, 25° C., for 10 min. Subsequently, 10 ml 1.7% (w/v) phosphoric acid was added to terminate the reaction and the mixture was centrifuged to remove precipitates. The amount of the alanyl glutamine product was determined: an enzyme level capable of producing 1 μmol alanyl glutamine every one minute under the specified reaction conditions was defined as 1 unit. The resulting alanyl glutamine was subjected to HPLC analysis under the following conditions: column=Inertsil ODS-2 (4.6×250 mm, supplied from GL Sciences); mobile phase=5 mM sodium 1-octanesulfonate/methanol=1000/165 (adjusted by phosphoric acid to pH2.1); flow rate=1.0 ml/min; temperature=40° C.; and UV detection made at 210 nm.

The ability of each E. coli transformant transformed with each of the respective plasmids to produce alanyl-glutamine was determined. The results are shown in Table 2 below. The pAF150-bearing transformant, in which native signal sequence had been substituted with the signal sequence of acid phosphatase, had 4.7 times as high an activity to produce alanyl-glutamine as the expression of the transformant bearing the plasmid pTrpT_Gtg2 containing trp promoter, proving that the substitution of the native signal sequence with the acid phosphatase-derived signal sequence significantly increases the expression level. When the acid phosphatase-derived signal sequence was integrated downstream of the acid phosphatase-derived promoter, the activity was further increased.

TABLE 2

| | | | Activity | |
|---|---|---|---|---|
| Strains | Promoter | Signal | (U/ml broth) | Factor |
| Escherichia coli | trp | WT | 0.60 | 1 |
| JM109/pTrpT_Gtg2 | | | | |
| Escherichia coli | trp | Acid | 2.80 | 4.7 |
| JM109/pAF150 | | phosphatase | | |
| | | signal | | |
| Escherichia coli | Acid | Acid | 5.54 | 9.2 |
| JM109/pAF110 | phosphatase- | phosphatase | | |
| | derived | signal | | |

### Example 3

### Construction of Plasmids Capable of High Level Expression of Arginine Racemase Derived from Pseudomonas taetrolens

(1) Isolation of Arginine Racemase Gene

Oligonucleotide PCR primers PRP1 and PRP2, designed to amplify the known nucleotide sequence of arginine racemase gene of Pseudomonas taetrolens (EMBL Accession number AB096176), were synthesized. A loopful cells of Pseudomonas taetrolens strain ATCC 4683 was inoculated into a 50 ml LB liquid medium in a 500 ml Sakaguchi flask and was cultivated by shake-culture at 30° C. while agitated. Subsequently, the culture solution was centrifuged (12,000 rpm, 4° C., 15 min) to recover the cells and chromosomal DNA was obtained by using QIAGEN Genomic-tip System (supplied from Qiagen) according to the manufacturer's instruction.

A PCR mixture was prepared by adding 0.1 ng of the chromosomal DNA as a template, along with 2.5 μmol each of oligonucleotides AR1 (5′-GGA ATT CCA TAT GCC CTT CTC CCG TAC CCT GC: SEQ ID NO: 23) and AR2 (5′-CGG GAT CCC TGA TCT TTC AGG ATT TTA GGG TTG: SEQ ID NO: 24) as primers and 2.5 units of Taq DNA polymerase (supplied from Takara), to a 100 μl 100 mM Tris-HCl buffer (pH8.3) containing 200M each of dATP, dCTP, dGTP and dTTP, 50 mM potassium chloride, and 1.5 mM magnesium chloride. PCR was performed by 30 cycles of 94° C. for 30 sec, 55° C. for 2 min, and 72° C. for 3 min. The reaction mixture was subjected to an agarose gel electrophoresis and the amplified DNA fragments were recovered with a Microspin column (supplied from Amersham Pharmacia Biotech). The DNA fragments were digested with Nde I and BamH I and were ligated to the Nde I-BamH I site of pET28a vector (supplied from Novatgen). E. coli strain JM109 was transformed with this ligation mixture and were selected a clone that had incorporated the desired plasmid from ampicillin-resistanct strains. This plasmid was designated as pArgr100. The transformant showed not only phosphatase activity on nucleoside, but also a phosphotransferase activity. For the expression of the arginine racemase gene cloned into the pET vector, competent cells of E. coli strain BL21 (supplied from Novagen) were transformed with the pArgr100 plasmid obtained from the transformant of E. coli strain JM109. In this manner, E. coli strain BL21/pArgr100 was constructed.

(2) Construction of Plasmids Capable of High Level Expression of Arginine Racemase Gene

Using PCR technique, an expression plasmid was constructed that includes the gene encoding mature protein of arginine racemase (peptide-synthesizing enzyme) integrated downstream of the modified promoter and the signal sequence of acid phosphatase derived from Enterobacter aerogenes. Specifically, the pArgr100 plasmid as a template, 0.4 mM each of oligonucleotides CAR1 (5′-TCC GTT AAC GCT TTC GCG GCG CCA CCC CTG TCG ATG ACC: SEQ ID NO: 25) and CAR2 (5′-TTG ACG TCT TAC TGA TCT TTC AGG ATT TTA GGG: SEQ ID NO: 26) as primers, a buffer for KOD plus (supplied from Toyobo), 0.2 mM each of dATP, dCTP, dGTP and dTTP, 1 mM magnesium sulfate, and 1 unit of KOD plus polymerase (supplied from Toyobo) were mixed together to form a 50 μl reaction mixture. The gene encoding mature protein of the peptide-synthesizing enzyme was amplified by performing PCR under the following conditions: an initial heat treatment at 94° C. for 30 sec, followed by 25 cycles of 94° C. for 15 sec, 55° C. for 30 sec, and 68° C. for 2 min and 30 sec. Meanwhile, using pEAP130 plasmid of Example 1 as a plasmid, along with the oligonucleotides of CAR3 (5′-GCT CTA GAA TTT TTT CAA TGT GAT TT: SEQ ID NO: 27) and CAR4 (5′-CAT CGA CAG GGG TGG CGC CGC GAA AGC GTT AAC GGA AAA C: SEQ ID NO: 28) as primers, promoter and the signal sequence region of acid phosphatase, amplification by PCR was performed under the same conditions as above. The reaction mixture was subjected to an agarose gel electrophoresis and the amplified DNA fragments were recovered with a Microspin column (supplied from Amersham Pharmacia Biotech).

Using a mixture of the amplified fragments as a template, along with the CAR2 and CAR3 oligonucleotides as primers, PCR was performed in a similar reaction mixture by 25 cycles of 94° C. for 15 sec, 55° C. for 30 sec, and 68° C. for 2 min and 30 sec. In this manner, a chimeric enzyme gene was constructed. The amplified DNA fragments were recovered with a Microspin column (supplied from Amersham Pharmacia Biotech) and were digested with Xba I and Pst I. The digest fragment was ligated to the Xba I-Pst I site of pUC19 plasmid (supplied from Takara) and pTWV229 plasmid (supplied from Takara). By using the Dye Terminator technique with DNA sequencing kit Dye Terminator Cycle Sequencing Ready Reaction (supplied from PERKIN ELMER), the nucleotide sequence of the plasmids were determined by a 310 Genetic analyzer (supplied from ABI), which confirmed that the desired recombinant gene had been obtained. The chimeric enzyme gene integrated into a plasmid pUC19 made a plasmid pArgr200, and the chimeric enzyme gene integrated into a plasmid pTWV229 made a plasmid pArgr300.

(3) Assessment of the Activity of Arginine Racemase in Transformants

Pseudomonas taetrolens strain ATCC 4683, E. coli strain BL21 transformed with a plasmid pArgr100, and E. coli strain JM109 transformed with a plasmid pArgr200 or pTWV229 were individually inoculated in a 3 ml LB liquid medium (10 g/l bacto tryptone, 5 g/l bacto yeast extract, and 10 g/l NaCl) and pre-cultivations were respectively carried out at 30° C. for 8 hours. 100 mg/l ampicillin was added to each culture of E. coli transformants.

Subsequently, a 2.5 ml portion resultant of each pre-cultivation was inoculated in a 50 ml arginine racemase expression medium (10 g/l yeast extract, 10 g/l tryptone, 5 g/l (NH₄)₂SO₄, 5 g/l KH₂PO₄, 1 g/l K₂HPO₄, 0.5 g/l MgSO₄.7H₂O, 0.01 g/l FeSO₄.7H₂O, 0.01 g/l MnSO₄.7H₂O, and 0.1 g/l thiamine hydrochloride) in a 500 ml Sakaguchi flask and shake-culture was carried out. The cells were cultivated at 30° C. for 20 hours. 100 mg/l ampicillin was added to each culture medium for E. coli transformants and 1 mM IPTG was added to each culture medium for E. coli strain BL21 transformants. After the cultivation period, the cells were recovered, were washed, and were assessed for the arginine racemase activity.

Using L-arginine as substrate, the arginine racemase activity was determined in the following manner: a reaction mixture (1 ml) was prepared containing 100 mg/l L-arginine, 200 μg/ml pyridoxal phosphate, 100 μmol/ml Tris-HCl buffer (pH8.0), and the cells. The reaction was then allowed to proceed at 30° C. for 10 min. Subsequently, 9 ml perchloric acid solution (pH1.0) was added to terminate the reaction and the resultant mixture was centrifuged to remove precipitates. The amount of the D-arginine product was determined: an enzyme level capable of producing 1 μmol D-arginine every one minute under the specified reaction conditions was defined as 1 unit. The resulting L-arginine and D-arginine were subjected to HPLC analysis under the following conditions: column=Crownpak (+) (4.0×150 mm, supplied from Daicel Chemical Industries); mobile phase=perchloric acid (pH1.0); flow rate=0.4 ml/min; temperature=40° C.; and UV detection made at 210 nm.

The arginine racemase activity in each E. coli transformant transformed with each of the respective plasmids was determined. The results are shown in Table 3 below. E. coli strain DE21/pArgr100 transformed with the pArgr100, which arginine racemase gene had been cloned into the pET vector, showed lower activity than the parent strain. In comparison, pArgr200- and pArgr300-bearing strains, in which native signal sequence had been substituted with the signal sequence of acid phosphatase, each exhibited more than 100 times higher activity than the parent strain. This indicates that the substitution of the native signal sequence with the acid phosphatase-derived signal sequence significantly increases the expression level.

TABLE 3

| | Signal | | Activity | |
|---|---|---|---|---|
| Strains | sequence | Promoter | (u/ml) | Factor |
| Pseudomonas | — | — | 1.27 | 1 |
| taetrolens ATCC4683 | | | | |
| Escherichia coli | WT | T7 | 0.55 | 0.43 |
| DE21/pArgr100 | | | | |
| Escherichia coli | Phosphatase- | Phosphatase- | 168 | 132 |
| JM109/pArgr200 | derived | derived | | |
| Escherichia coli | Phosphatase- | Phosphatase- | 187 | 147 |
| JM109/pArgr200 | derived | derived | | |

Although the present invention has been described with reference to the preferred examples, it should be understood that various modifications and variations can be easily made by those skilled in the art without departing from the spirit of the invention. Accordingly, the foregoing disclosure should be interpreted as illustrative only and is not to be interpreted in a limiting sense. The present invention is limited only by the scope of the following claims along with their full scope of equivalents.

Free Text in the Sequence Listing

SEQ ID NO: 1; amino acid sequence of signal peptide
SEQ ID NO: 2; nucleotide sequence of signal peptide
SEQ ID NO: 3; amino acid sequence of peptide-synthesizing enzyme (mature protein)
SEQ ID NO: 4; amino acid sequence of arginine racemase
SEQ ID NO: 5; Primer EM1
SEQ ID NO: 6; Primer EMR1
SEQ ID NO: 7; amino acid sequence determined by Edman degradation of peptide-synthesizing protein derived from Empedobacter
SEQ ID NO: 8; amino acid sequence determined by Edman degradation of peptide-synthesizing protein derived from Empedobacter
SEQ ID NO: 9; Mix primer
SEQ ID NO: 10; Mix primer
SEQ ID NO: 11; nucleotide sequence of peptide-synthesizing protein derived from Empedobacter brevis
SEQ ID NO: 12; amino acid sequence of peptide-synthesizing protein derived from Empedobacter brevis
SEQ ID NO: 13; primer for preparation of pTrpT
SEQ ID NO: 14; primer for preparation of pTrpT
SEQ ID NO: 15; primer for preparation of pTrpT_Gtg2
SEQ ID NO: 16; primer for preparation of pTrpT_Gtg2
SEQ ID NO: 17; Primer CAP1
SEQ ID NO: 18; Primer CAP2
SEQ ID NO: 19; Primer CAP3
SEQ ID NO: 20; Primer CAP4
SEQ ID NO: 21; Primer CAP5
SEQ ID NO: 22; Primer CAP6
SEQ ID NO: 23; Primer AR1
SEQ ID NO: 24; Primer AR2
SEQ ID NO: 25; Primer CAR1
SEQ ID NO: 26; Primer CAR2
SEQ ID NO: 27; Primer CAR3
SEQ ID NO: 28; Primer CAR4

1. A recombinant polynucleotide, comprising a nucleotide sequence that encodes a signal peptide derived from the N-terminal region of acid phosphatase present in bacterium belonging to Enterobacteriaceae.

2. The recombinant polynucleotide according to claim 1, wherein the bacterium belonging to Enterobacteriaceae is selected from the group consisting of Enterobacter aerogenes, Escherichia blattae, Klebsiella planticola, Morganella morganii, Providencia stuartii, and Shigella flexneri.

3. A recombinant polypeptide, comprising a nucleotide sequence that encodes the signal peptide of SEQ ID NO: 1 in the sequence listing.

4. The recombinant polynucleotide according to claim 1, comprising a nucleotide sequence encoding a protein integrated downstream of the nucleotide sequence encoding the signal peptide.

5. The recombinant polynucleotide according to claim 4, wherein the protein is selected from the group consisting of:
(A) a protein having the amino acid sequence of SEQ ID NO:3 in the sequence listing; and
(B) a protein having a peptide-synthesizing activity and having the amino acid sequence that includes one or several amino acids mutation in the amino acid sequence of SEQ ID NO: 3, the mutation being selected from the group consisting of substitution, deletion, insertion, addition, and inversion.

6. The recombinant polynucleotide according to claim 4, wherein the protein is selected from the group consisting of:
(C) a protein having the amino acid sequence of SEQ ID NO:4 in the sequence listing; and
(D) a protein having an arginine racemase activity and having the amino acid sequence that includes one or several amino acids mutation in the amino acid sequence of SEQ ID NO: 4, the mutation being selected from the group consisting of substitution, deletion, insertion, addition, and inversion.

7. A transformant, comprising a recombinant polynucleotide that contains a nucleotide sequence encoding a signal peptide derived from the N-terminal region of acid phosphatase present in bacterium belonging to Enterobacteriaceae and contains a nucleotide sequence encoding a protein integrated downstream of the nucleotide sequence encoding the signal peptide.

8. A transformant according to claim 7, wherein the bacterium belonging to Enterobacteriaceae is selected from the group consisting of Enterobacter aerogenes, Escherichia blattae, Klebsiella planticola, Morganella morganii, Providencia stuartii, and Shigella flexneri.

9. A transformant according to claim 7,
wherein the protein is selected from the group consisting of:
(A) a protein having the amino acid sequence of SEQ ID NO:3 in the sequence listing; and
(B) a protein having a peptide-synthesizing activity and having the amino acid sequence that includes one or several amino acids mutation in the amino acid sequence of SEQ ID NO: 3, the mutation being selected from the group consisting of substitution, deletion, insertion, addition, and inversion.

10. A transformant according to claim 7,
wherein the protein is selected from the group consisting of:
(C) a protein having the amino acid sequence of SEQ ID NO:4 in the sequence listing; and
(D) a protein having an arginine racemase activity and having the amino acid sequence that includes one or several amino acids mutation in the amino acid sequence of SEQ ID NO: 4, the mutation being selected from the group consisting of substitution, deletion, insertion, addition, and inversion.

11. A transformant, comprising a recombinant polynucleotide that contains a nucleotide sequence encoding a signal peptide of SEQ ID NO: 1 in the sequence listing and contains a nucleotide sequence encoding a protein integrated downstream of the nucleotide sequence encoding the signal peptide.

12. A transformant according to claim 11,
wherein the protein is selected from the group consisting of:
(A) a protein having the amino acid sequence of SEQ ID NO:3 in the sequence listing; and
(B) a protein having a peptide-synthesizing activity and having the amino acid sequence that includes one or several amino acids mutation in the amino acid sequence of SEQ ID NO: 3, the mutation being selected from the group consisting of substitution, deletion, insertion, addition, and inversion.

13. A transformant according to claim 11,
wherein the protein is selected from the group consisting of:
(C) a protein having the amino acid sequence of SEQ ID NO:4 in the Sequence Listing; and
(D) a protein having an arginine racemase activity and having the amino acid sequence that includes one or several amino acids mutation in the amino acid sequence of SEQ ID NO: 4, the mutation being selected from the group consisting of substitution, deletion, insertion, addition, and inversion.

14. A production method of a protein, wherein the transformant of claim 7 is cultivated in a growth medium, so that the protein accumulates in the growth medium and/or in the microbial cells.

15. A production method of a protein, wherein the transformant of claim 8 is cultivated in a growth medium, so that the protein accumulates in the growth medium and/or in the microbial cells.

16. A production method of a protein, wherein the transformant of claim 9 is cultivated in a growth medium, so that the protein accumulates in the growth medium and/or in the microbial cells.

17. A production method of a protein, wherein the transformant of claim 10 is cultivated in a growth medium, so that the protein accumulates in the growth medium and/or in the microbial cells.

18. A production method of a protein, wherein the transformant of claim 11 is cultivated in a growth medium, so that the protein accumulates in the growth medium and/or in the microbial cells.

19. A production method of a protein, wherein the transformant of claim 12 is cultivated in a growth medium, so that the protein accumulates in the growth medium and/or in the microbial cells.

20. A production method of a protein, wherein the transformant of claim 13 is cultivated in a growth medium, so that the protein accumulates in the growth medium and/or in the microbial cells.

21. The recombinant polynucleotide according to claim 3, comprising a nucleotide sequence encoding a protein integrated downstream of the nucleotide sequence encoding the signal peptide.

22. The recombinant polynucleotide according to claim 21, wherein the protein is selected from the group consisting of:
(A) a protein having the amino acid sequence of SEQ ID NO:3 in the sequence listing; and
(B) a protein having a peptide-synthesizing activity and having the amino acid sequence that includes one or several amino acids mutation in the amino acid sequence of SEQ ID NO: 3, the mutation being selected from the group consisting of substitution, deletion, insertion, addition, and inversion.

23. The recombinant polynucleotide according to claim 21, wherein the protein is selected from the group consisting of:
(C) a protein having the amino acid sequence of SEQ ID NO:4 in the sequence listing; and
(D) a protein having an arginine racemase activity and having the amino acid sequence that includes one or several amino acids mutation in the amino acid sequence of SEQ ID NO: 4, the mutation being selected from the group consisting of substitution, deletion, insertion, addition, and inversion.
